(19) United States
(12) Patent Application Publication
(10) Pub. No.: US 2007/0080081 A1
(43) Pub. Date: Apr. 12, 2007

(54) Toothbrush Box With Sterilization Function

(76) Inventor: Wu-Lang Chang, Chung-Li City (TW)

Correspondence Address: Lin & Associates Intellectual Property, Inc., P. O. Box 2339, Saratoga, CA, 95070-0339, US

(21) Appl. No.: 11/538,057

(22) Filed: Oct. 3, 2006

(30) Foreign Application Priority Data
Oct. 6, 2005 (TW) 094217306

(51) Int. Cl. B65D 83/10

(52) U.S. Cl. 206/362

(57) ABSTRACT

A toothbrush box with sterilization function includes a box body, a fixing rack, an ozone generator, a negative ion generator, an ultraviolet lamp, and an antibiotic filter that is coated with a nano-silver on a surface thereof and is wrapped on the ultraviolet lamp, wherein the fixing rack and the ultraviolet lamp are mounted on an upper surface of a bottom portion of the box body, and the ozone generator and the negative ion generator are installed in a compartment of the bottom portion. When the toothbrush box is power supplied, the ultraviolet lamp wrapped with the antibiotic filter, the ozone generator and the negative ion generator can sterilize toothbrushes placed in the box. Moreover, the negative ion generator can produce negative ions to dehumidify and deodorize the toothbrushes.

### BACKGROUND OF THE INVENTION

1. Field of the Invention

The present invention relates generally to a toothbrush box with sterilization function, and in particular to a toothbrush box for sterilization by using an ozone generator, a negative ion generator and an ultraviolet lamp wrapped with an antibiotic filter.

2. The Prior Arts

Tooth-brushing is an indispensable routine activity in average person's daily life. Each person in his/her early age is taught to develop a habit of cleaning the teeth when he/she gets in and out of bed. Some families who pay great attention to the hygiene request the child to clean the teeth after three meals to maintain the teeth clean, not only for maintaining the oral cavity healthy, but for maintaining the teeth pleasing as well. A person who develops the good habit of frequent teeth cleaning has less probability to get dental caries and periodontal disease in the future days.

It is obvious that brushing teeth is a good habit for maintaining oral cavity clean. However, a toothbrush is a tool that people always neglect to take care of, especially the bristles where bacterium and virus are easily grown and reproduced. Even if the toothbrush is washed after it is used to clean the dregs of foods or the tartar between the teeth, slight residue will still remain on the bristles that cause the reproduction of the bacteria. To solve the aforementioned problem, lots of toothbrush disinfectants and new toothbrush designs are proposed to reduce the possibility of bacterium growth.

Generally speaking, using disinfectants to periodically clean the toothbrush wastes time and money, and also leaves the worry that the disinfectant might be retained in the bristles. In addition, users are frequently forgotten to sterilize the toothbrush by using disinfectants during busy days, and thus the method to clean the toothbrush is seldom adopted. Due to the drawback and inconvenience of the disinfectants, manufacturers proposed a new toothbrush with a bristle made of nanometer material that can kill the bacteria. Such a toothbrush particularly fits the busy office workers. However, it is understood that the so-called bristles with sterilization function only sustain a few weeks. The effect of killing bacteria decreases day by day. As a result, consumers are fooled by the ads, spending more money only to get less effect. Thus, it is desired to invent a toothbrush box with sterilization function.

### SUMMARY OF THE INVENTION

A primary objective of the present invention is to provide a toothbrush box with sterilization function by means of a sterilization lamp with a nano-metal attached thereon, and the ozone and negative ion generators provided in the box. When a toothbrush is placed in the box, a bristle can be sterilized to prevent the bacteria from growth and reproduction, such that the bacteria infection could be avoided.

Another objective of the present invention is to provide a box body to receive a toothbrush so as to prevent the toothbrush from exposing in the air and contacting with many dusts, which result in the elasticity lose of the bristles. When the toothbrush is received in the box, it will remain new and clean even if it has not been used for a period of time. In addition, a bathroom can be decorated by the design and colorful pattern of the box body.

To achieve the above-mentioned objectives, a toothbrush box in accordance with the present invention includes a plastic box body and a cover covered on the box body. The cover pivotally connects to the box body by a bolt; hence the box could be opened freely. A fixing rack having a plurality of grooves for fixing toothbrushes in position is integrally formed or fastened on an upper surface of a bottom portion of the box body. A sterilization lamp is located on the upper surface of the bottom portion close to a bristle of the toothbrush after the toothbrush is placed on the fixing rack, and an antibiotic filter is wrapped on a surface of the sterilization lamp. When the toothbrush is placed on the fixing rack, the ultraviolet lamp can sterilize the bristles effectively and conveniently. An ozone generator and a negative ion generator are installed in a compartment defined in the bottom portion of the box body. When the box is closed, the generated ozone and negative ions can purify the air in the box to inhibit the bacteria from growth and reproduction on the toothbrush. In addition, the negative ions have a function of dehumidification, which can dehumidify the toothbrush to prevent it from being moldy and stinky.

The toothbrush box in accordance with the present invention can be provided for a whole family use, in which the fixing rack having a plurality of the grooves can provide several toothbrushes to place thereon to perform the sterilization together, which is very practical and economical.

Moreover, a bottom of the toothbrush box of the present invention further has a plurality of suction discs provided on a lower surface of the bottom portion, so that the toothbrush box can be attached on a tile, a glass or a mirror. It is a convenient design since the toothbrush box is always installed in a bathroom, and such design can prevent from drilling holes on a wall for installation and can be conveniently installed on the place near where an electric socket is located.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective view showing a toothbrush box in accordance with the present invention.

FIG. 2 is a perspective view showing that a plurality of toothbrushes are placed in the toothbrush box of FIG. 1.

FIG. 3 is a cross-sectional view showing that the toothbrush box of FIG. 2 is mounted on a wall.

FIG. 4 is an exploded view showing a sterilization lamp and an antibiotic filter of the toothbrush box in accordance with the present invention.

FIG. 5 is an assembled view of FIG. 4.

FIG. 6 is a cross-sectional view of FIG. 5.

FIG. 7 is an assembled view showing a sterilization lamp and an antibiotic filter of a toothbrush box in accordance with another embodiment of the present invention.

FIG. 8 is an exploded view showing a toothbrush box in accordance with another embodiment of the present invention.

### DETAILED DESCRIPTION OF THE PREFERRED EMBODIMENT

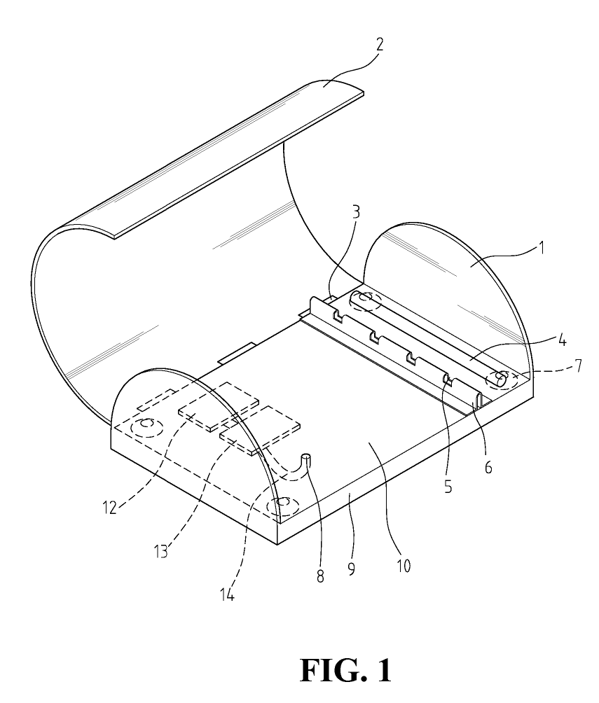

With reference to FIG. 1, a toothbrush box in accordance with the present invention comprises a plastic box body 1 and a cover 2 covered on the box body 1. The cover 2 pivotally connects to the box body 1 by a bolt 3; hence the box could be opened and closed freely. A fixing rack 6 is mounted on an upper surface 10 of a bottom portion of the box body 1 and has a plurality of grooves 5 for placing toothbrushes by fitting tightly a handle of each toothbrush into the grooves 5. A sterilization lamp 4 wrapped with an antibiotic filter is located on the upper surface 10 of the bottom portion of the box body 1 close to the bristle of the toothbrush after the toothbrush is placed on the fixing rack 6. A compartment 9 is defined in the bottom portion of the box body 1, and an ozone generator 12 and a negative ion generator 13 are installed in the compartment 9. A transmission pipe 14 is connected to the ozone generator 12 and the negative ion generator 13 for delivering ozone and negative ions to a vent 8 on the upper surface 10 of the bottom portion of the box body 1. Moreover, a lower surface of the bottom portion of the box body 1 has a plurality of suction discs 7 thereon, such that the toothbrush box can be attached to a tile, a glass or a mirror.

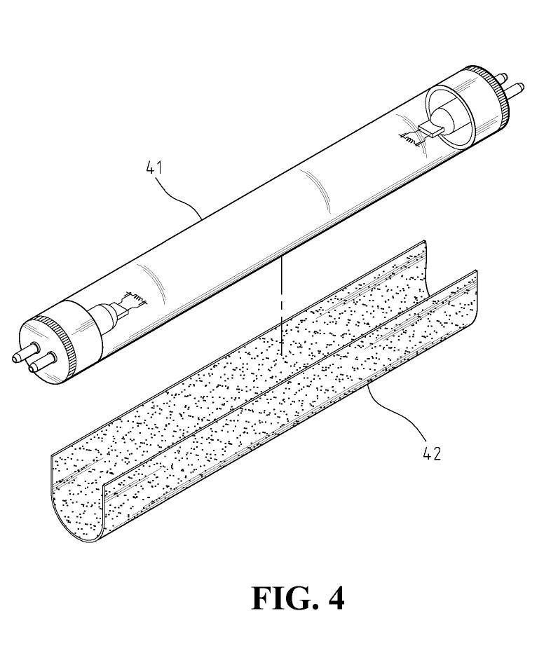

Referring to FIGS. 4-6, the sterilization lamp 4 may be an ultraviolet lamp 41 or a nano-lamp, and an antibiotic filter 42 is made of fibers and wrapped on a surface of the ultraviolet lamp 41. The fibers can be paper or fabric material. The sterilization capability of the toothbrush box is thus enhanced by combination of the antibiotic filter 42 and the sterilization lamp 4.

The ultraviolet lamp 41 can emit ultraviolet ray with a wavelength of 260 nm which has a greater capacity to kill a genetic material (DNA and RNA) of a virus or a bacteria. A microbe is unable to grow and reproduce because the genetic material of the microbe will be sabotaged under the radiation of the ultraviolet lamp 41 for a certain period of time, and thereby the objective of sterilization can be achieved as well.

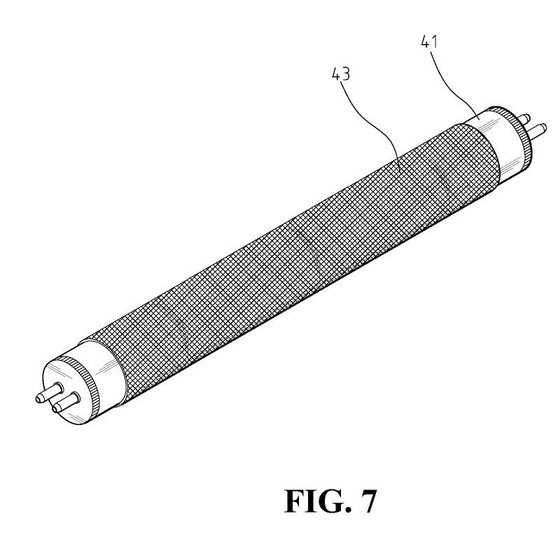

The antibiotic filter 42 may also be made of woven mesh 43 (see FIG. 7). A nano-metal, such as nano-silver, with sterilization and deodorization function may be coated in a laminated structure or dispersedly in a granulated structure on a surface of the antibiotic filter 42.

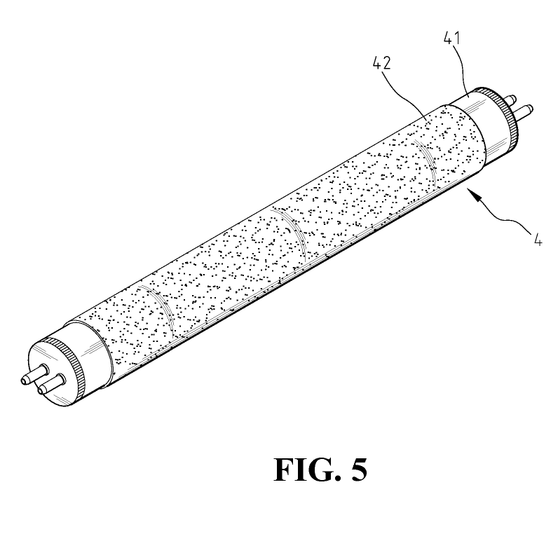

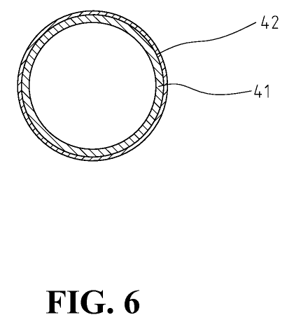

Referring to FIGS. 5 and 6, the antibiotic filter 42 coated with the nano-silver is wrapped on or sleeved onto the surface of the ultraviolet lamp 41. When the foul air containing bacteria passes across the ultraviolet lamp 41, the nano-silver and the ultraviolet lamp 41 will deodorize the foul air and kill the bacteria quickly and effectively.

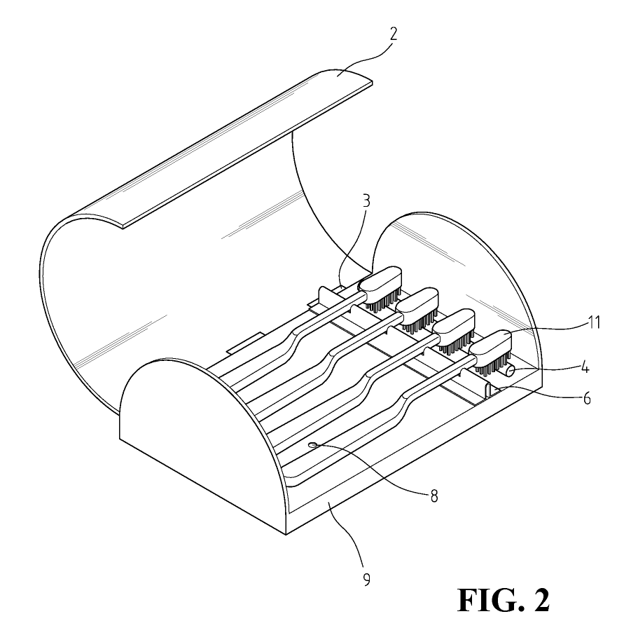

As shown in FIG. 2, toothbrushes 11 are placed in the box and fixed on the fixing rack 6 with the bristle faced downwardly and the handles of the toothbrushes 11 are tightly fitted into the grooves 5, whereby the bristles of the toothbrushes 11 can be sterilized effectively by the sterilization lamp 4 wrapped with the antibiotic filter 42 that is disposed on the upper surface 10 of the bottom portion of the box body 1 close to the bristles. When the cover 2 is closed, the toothbrush box forms a sealed space so that the ozone generator 12 and the negative ion generator 13 installed in the compartment 9 start to deliver the ozone and negative ions to the vent 8 through the transmission pipe 14 to purify the inner air of the box. In addition, the negative ions have a function of dehumidification, which can dehumidify the toothbrushes to prevent them from being moldy when the box is closed.

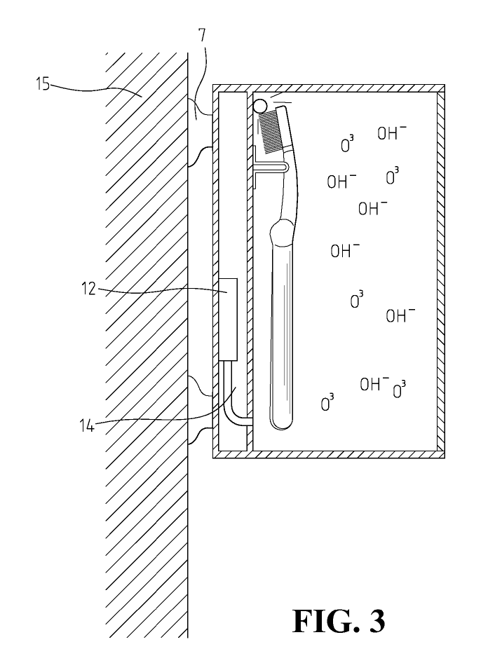

FIG. 3 shows that the toothbrush box in accordance with an embodiment of the present invention is hanged on a wall 15. The toothbrush box can be attached to the wall 15 in a bathroom by the sucking of the suction discs 7.

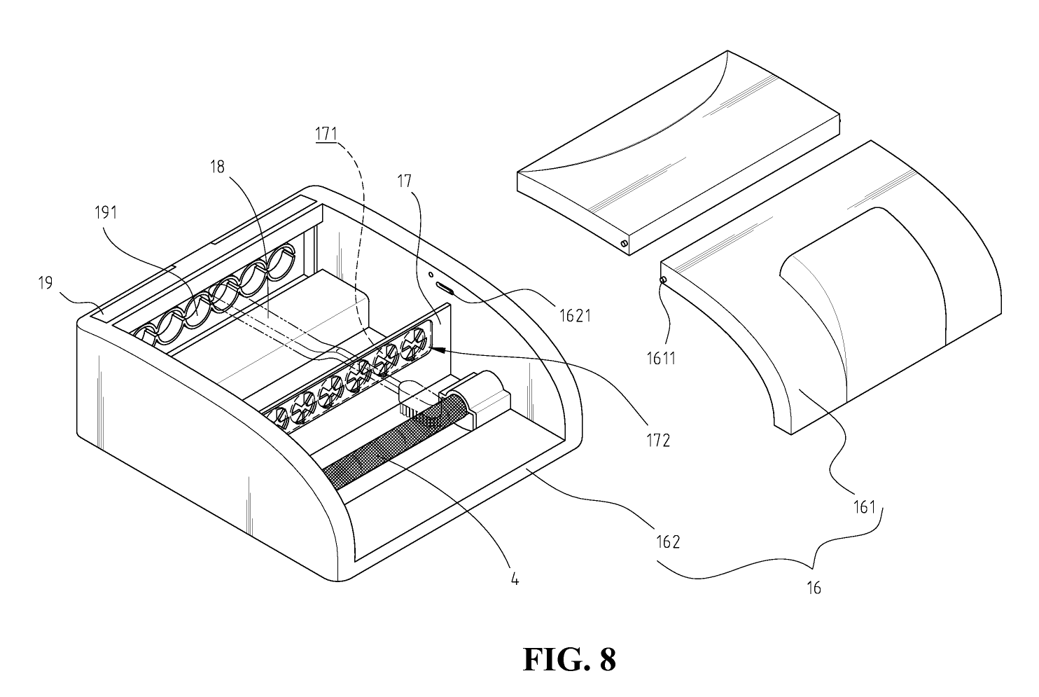

FIG. 8 shows a toothbrush box in accordance with another embodiment of the present invention. A partition board 17 is disposed in a toothbrush box 16 in a direction vertical to an upper surface of a bottom portion of a box body 162. The partition board 17 has a plurality of holes 171 defined therethrough. A soft pad 172, which is formed in a petal shape and made of plastic or rubber material, is provided in an inner rim of each hole 171. A center of each soft pad 172 defines a passage for passing a handle of a toothbrush 18, in which the bristles can be positioned and face correspondingly to the sterilization lamp 4, and thus sterilization can be performed when the sterilization lamp 4 is on. A rear board 19, which is set parallel to the partition board 17, is able to be withdrawn from the box body 16 and has an end shelf 191 mounted on one side thereof for placing a rear end of the handle of the toothbrush 18. When the rear end of the handle is placed on the end shelf 191, the toothbrush 18 is positioned in inclined status, which makes the water remained in the bristles drain out along the handle to the end shelf 191. The rear board 19 can be withdrawn periodically to pour away the water therein. The installation manner of the cover 161 and the box body 162 in accordance with the present embodiment is different from that of the first embodiment. As for this embodiment, an inner surface of both side walls of the box body 162 respectively has a short groove 1621 defined thereon, and both sides of the cover 161 each have a protruded post 1611 for being slidably inserted in the groove 1621. When the protruded post 1611 is pushed to a rear end of the groove 1621, the cover 161 is closed. In contrast, when the protruded post 1611 is pushed to a front end of the groove 1621, the cover 161 can be opened by the rotation of protruded post 1611 in the front end of the groove 1621.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A toothbrush box with sterilization function, comprising
a box body having a cover and a bottom portion with a compartment provided in the bottom portion, wherein an ozone generator and a negative ion generator are installed in the compartment;
a fixing rack mounted on an upper surface of the bottom portion and having a plurality of grooves for fixing a toothbrush in position;
a sterilization lamp located on the upper surface of the bottom portion close to a bristle of the toothbrush after the toothbrush is placed on the fixing rack;
an antibiotic filter wrapped on the sterilization lamp and having a nano-metal coated on a surface thereof.

2. The toothbrush box as claimed in claim 1, wherein the sterilization lamp is an ultraviolet lamp.

3. The toothbrush box as claimed in claim 1, wherein the sterilization lamp is a nano-lamp.

4. The toothbrush box as claimed in claim 1, wherein the nano-metal is nano-silver.

5. The toothbrush box as claimed in claim 1, wherein the nano-metal is coated in a laminated structure on the surface of the antibiotic filter.

6. The toothbrush box as claimed in claim 1, wherein the nano-metal is dispersedly coated in a granulated structure on the surface of the antibiotic filter.

7. The toothbrush box as claimed in claim 1, wherein the antibiotic filter is made of woven mesh.

8. The toothbrush box as claimed in claim 1, wherein the antibiotic filter is made of fibers.

9. The toothbrush box as claimed in claim 8, wherein the fibers are fabric material.

10. The toothbrush box as claimed in claim 8, wherein the fibers are paper material.

11. The toothbrush box as claimed in claim 1, wherein a plurality of suction discs are provided on a lower surface of the bottom portion.
